(19) United States
(12) Patent Application Publication
(10) Pub. No.: US 2006/0067962 A1
(43) Pub. Date: Mar. 30, 2006

(54) Skin cooling compositions

(75) Inventors: Duane G. Krzysik, Appleton, WI (US); Katherine D. Stahl, Appleton, WI (US); Julie M. Utschig, Appleton, MI (US); Jamie Lynn Sondelski, Wausau, WI (US); James A. Cotter II, Waupaca, WI (US); Bruce M. Siebers, Kimberly, WI (US); Daniel J. Heuer, Fremont, WI (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(73) Assignee: Kimberly-Clark Worldwide, Inc.

(21) Appl. No.: 11/027,635

(22) Filed: Dec. 28, 2004

Related U.S. Application Data
(63) Continuation-in-part of application No. 10/957,506, filed on Sep. 30, 2004.

(51) Int. Cl. A61K 8/89

(52) U.S. Cl. 424/401; 424/70.12

(57) ABSTRACT

The present invention relates to an applicator and method for cooling skin. The applicator includes a substrate and a skin cooling composition that is attached to the substrate. In some embodiments, the skin cooling composition includes water, alcohol, a surfactant and a water dispersible silicone. As used herein, a water dispersible silicone includes a water dispersible silicone and/or a water soluble silicone. In other embodiments, the skin cooling composition includes water, a cooling agent and a polymeric emulsifier. The method of cooling skin includes positioning an applicator near the skin where the applicator includes a substrate and a skin cooling composition as described herein that is attached to the substrate. The method further includes engaging the substrate with the skin to apply the skin cooling composition to the skin.

### CROSS REFERENCE TO RELATED APPLICATION

This application is a Continuation-In-Part of U.S. application Ser. No. 10/957,506 filed Sep. 30, 2004.

### FIELD OF THE INVENTION

The invention relates to an applicator and method for cooling skin, and in particular to an applicator and method that provides short and long term skin cooling.

### BACKGROUND OF THE INVENTION

There are a variety of products that are applied to the skin. It would be desirable under many circumstances if such products provided a cooling feeling to the skin when the products were applied to the skin. Some example products include lotions, creams, moisturizers, bath agents and insect-repellent sprays (among others).

Existing products typically provide skin cooling by combining skin cooling agents with other substances. However, many existing products fail to provide satisfactorily strong and long lasting skin cooling.

One example cooling agent is menthol which provides cooling in the form of a physiological effect on nerve endings in the human body that sense temperature. The cooling effect from menthol is not due to latent heat of evaporation but appears to be the result of direct stimulus on the cold receptors at the nerve endings.

One of the drawbacks with products that include menthol is that the products typically have a strong mint odor and are relatively volatile. In addition, menthol can be irritating to the skin. Several other compounds are known to provide cooling but many of these compounds also exhibit an odor and/or volatility.

There is a need for skin cooling compositions that provide a refreshing feeling during or after use of the product. The skin cooling compositions should provide cooling strength and persistence while also being compatible with other agents that may be used in various skin products.

### SUMMARY OF THE INVENTION

The present invention relates to skin cooling compositions and methods for preparing skin cooling compositions. The skin cooling compositions provide immediate cooling of the skin as well as longer term cooling of the skin.

In some embodiments, the invention relates to a skin cooling composition that includes water, alcohol, a surfactant and a water dispersible silicone. Some example silicones include an ethoxylated dimethicone (PEG (X) dimethicone) with the following structures: 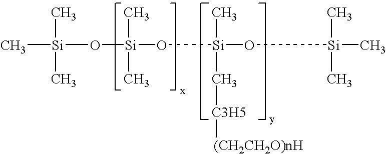
Where x=1 to about 500, y=1 to about 100 and n=1 to about 30.

As examples, the skin cooling composition may include an amount of the silicone between 1% and 5% by weight, an amount of the surfactant between 1% and 5% by weight, an amount of the alcohol between 15% and 45% by weight and an amount of the water between 50% and 85% by weight based on a total amount of the skin cooling composition.

In other embodiments, the invention relates to a skin cooling composition that includes water, a cooling agent and a polymeric emulsifier. As examples, the skin cooling composition may include an amount of the water between 80% and 95% by weight, an amount of the cooling agent between 1% and 5% by weight and an amount of the polymer emulsifier between 1% and 5% by weight based on a total amount of the skin cooling composition.

In still other embodiments, the invention relates to a skin cooling composition that includes water, alcohol, a cooling agent, a polymeric emulsifier and a water dispersible silicone. Some example silicones include an ethoxylated dimethicone (PEG (X) dimethicone) with the following structures: 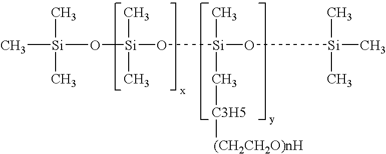
Where x=1 to about 500, y=1 to about 100 and n=1 to about 30.

As examples, the skin cooling composition may include an amount silicone between 1% and 5% by weight, an amount alcohol between 15% and 45% by weight, an amount water between 50% and 85% by weight, an amount cooling agent between 1% and 5% by weight, and an amount polymeric emulsifier between 1% and 5% by weight based on a total amount of the skin cooling composition.

In other embodiments, the invention relates to a method of forming a cooling composition. The method includes mixing water and alcohol to form a first mixture and mixing a solubilizing surfactant with vitamin E and silicone to form a second mixture. The method further includes adding a derivative of lactic acid to the first mixture to form a third mixture and mixing the third mixture with the second mixture to form the cooling composition. The method may further include adjusting the pH of the cooling composition to about 5.5 with citric acid.

In still other embodiments, the invention relates to another method of forming a cooling composition. The method includes mixing vitamin C with a derivative of lactic acid and a solubilizing surfactant to form a first mixture and mixing a cooling agent with vitamin E and a polymeric emulsifier to form a second mixture. The method further includes mixing the first mixture with the second mixture to form a third mixture and mixing water with a neutralizing base and a preservative to form a fourth mixture. The method further includes mixing the fourth mixture with the third mixture to form the cooling composition.

The present invention also relates to an applicator. The applicator includes a substrate and a skin cooling composition that is attached to the substrate. The skin cooling composition includes water, alcohol, a surfactant and a water dispersible silicone. It should be noted that the skin cooling composition may be similar to any of the cooling compositions described above which include a water dispersible silicone.

In another embodiment, the applicator includes a substrate and a skin cooling composition that is attached to the substrate. The skin cooling composition includes water, a cooling agent and a polymeric emulsifier. It should be noted that the skin cooling composition may be similar to any of the skin cooling compositions described above which include a polymeric emulsifier.

In yet another embodiment, the applicator includes a substrate that has a skin cooling composition which includes water, alcohol, a surfactant and a water dispersible silicone. It should be noted that the skin cooling composition may be similar to any of the skin cooling compositions described above which include a water dispersible silicone.

In yet another embodiment, the applicator includes a substrate that has a skin cooling composition which includes water, a cooling agent and a polymeric emulsifier. It should be noted that the skin cooling composition may be similar to any of the skin cooling compositions described above which include a polymeric emulsifier.

The present invention also relates to method of cooling skin. The method includes positioning an applicator near the skin where the applicator includes a substrate and a skin cooling composition that is attached to the substrate. The skin cooling composition includes water, alcohol, a surfactant and a water dispersible silicone. The method further includes engaging the substrate with the skin to apply the skin cooling composition to the skin. In some embodiments, engaging the substrate with the skin includes adhering the substrate to the skin and/or rubbing the substrate against the skin.

In another embodiment, the method includes positioning an applicator near the skin where the applicator includes a substrate and a skin cooling composition that is attached to the substrate. The skin cooling composition includes water, a cooling agent and a polymeric emulsifier. The method further includes engaging the substrate with the skin to apply the skin cooling composition to the skin.

In still another embodiment, the method includes positioning an applicator near the skin where the applicator includes a substrate having a skin cooling composition. The skin cooling composition includes water, alcohol, a surfactant and a water dispersible silicone. The method further includes engaging the substrate with the skin to apply the skin cooling composition to the skin.

In yet another embodiment, the method includes positioning an applicator near the skin where the applicator includes a substrate having a skin cooling composition. The skin cooling composition includes water, a cooling agent and a polymeric emulsifier. The method further includes engaging the substrate with the skin to apply the skin cooling composition to the skin. In some embodiments, engaging the substrate with the skin may include adhering the substrate to the skin. It should be noted that engaging the substrate with the skin may also include rubbing the substrate against the skin.

### BRIEF DESCRIPTION OF THE DRAWINGS

FIG. 1 is a perspective view illustrating an example system for cooling skin when the system is applied to skin.

FIG. 2 is a section of view of the system shown in FIG. 1 taken along line 2-2.

FIG. 3 is a section view similar to FIG. 2 illustrating another example system for cooling skin.

FIG. 4 is a section view similar to FIGS. 2-3 illustrating another example system for cooling skin.

FIG. 5 is a flow diagram illustrating an example method of cooling skin.

FIG. 6 is a flow diagram illustrating another example method of cooling skin.

FIG. 7 is a flow diagram illustrating still another example method of cooling skin.

FIG. 8 is a flow diagram illustrating yet another example method of cooling skin.

### DEFINITIONS

Within the context of this specification, each term or phrase below will include the following meaning or meanings:
(a) “Silicone” refers to a water dispersible silicone. As used herein, a water dispersible silicone includes water dispersible silicones and/or water soluble silicones. One example silicone includes an ethoxylated dimethicone (PEG (X) dimethicone) that conforms to the structure: 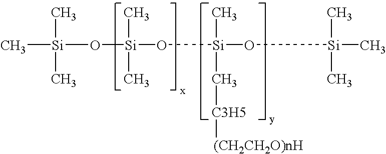
   Where x=1 to about 500, y=1 to about 100 and n=1 to about 30.
(b) “Water dispersible” silicones refers to silicone polymers that are either water soluble or capable of existing as stable colloidal, self-emulsifiable or other type dispersion in water and hydro-alcoholic solutions without the presence of added emulsifiers. Added emulsifiers may be employed within the scope of the present invention to assist in compatiblizing the polymers with other chemical agents used in the composition. However, the added emulsifiers may not be essential to formation of stable dispersions or solutions of the polymer in water.
(c) “Surfactant”, or “surface active agent”, includes compounds that reduce the surface tension when dissolved in an aqueous media. Surfactant also includes compounds that reduce the surface tension between incompatible or partially incompatible liquid phases, or between a liquid and a solid. Surfactants may function as detergents, emulsifiers, wetting agents and/or suspending agents.
(d) “Hydrophilic Lipophilic Balance”, or “HLB”, refers to the balance between the lipophilic tail portion and the hydrophilic head group in a surfactant (or combination of surfactants).
(e) “Alcohol” refers isopropyl alcohol and ethyl alcohol.
(f) “Cooling agent” refers to a material that produces a perception of an immediate and/or long term cooling sensation on skin when the material is applied to skin.
(g) “Polymeric emulsifier” refers to a cross linked acrylic acid polymer containing alkyl groups and/or acrylamide groups which aid in compatibility with organic oils. The polymer in the polymeric emulsifier may have varying molecular weights to control the viscosity of a cooling composition that includes the polymeric emulsifier.

### DETAILED DESCRIPTION OF THE INVENTION

The invention relates to a skin cooling composition that provides short and long term skin cooling. The composition is non-toxic and readily applied to the skin. The composition may also be incorporated into many skin-related products such that the products provide a refreshing feeling on the skin of individuals that use the products. In addition, the composition may be readily mixed with many other materials that can be included in products which are applied to the skin.

Silicone Composition

In some embodiments, the cooling composition includes a water dispersible silicone. As used herein, water dispersible silicone includes water dispersible silicones and/or water soluble silicones. One example water dispersible silicone includes ethoxylated dimethicone (PEG (X) dimethicone) that conforms to the structure: 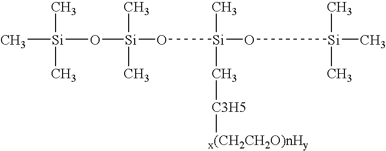
Where x=1 to about 500, y=1 to about 100 and n=1 to about 30.

The cooling composition further includes water and alcohol that provide the cooling composition with some of the short and long term cooling of the skin through the latent heat of evaporation from the alcohol (shorter term cooling) and water (longer term cooling). In some embodiments, the water may be purified or distilled. In addition, the alcohol may be isopropyl alcohol or ethyl alcohol.

The percentage of water and alcohol in the cooling composition may vary depending on the desired application of the cooling composition. As an example, the cooling composition may include about 50% to about 80% water. In other embodiments, the cooling composition may include about 50% to about 70%, or about 60% to about 80%, or about 60% to about 70% water. In addition, the cooling composition may include about 15% to about 45% alcohol. In other embodiments, the cooling composition may include about 15% to about 30%, or about 30% to about 45%, or about 20% to about 30% alcohol.

The cooling composition further includes a surfactant that may promote emulsifying activity. Surfactants have the ability to lower the surface tension of water to reduce the interfacial tension between two immiscible substances. In some embodiments, the surfactants in the cooling composition may enhance cleaning or removal of dirt, sweat, and/or sebum from the skin. Some surfactants may also act as a wetting agent to facilitate placing the cooling composition on a substrate (e.g., a wipe). In addition, some surfactants may act as emulsifying agents or solubilizing agents to emulsify or solublize hydrophobic materials into hydro-alcohol formulations.

As an example, the surfactant in the cooling composition may have an HLB of about 6 to about 18. In addition, a combination of surfactants may be used to achieve a desired HLB.

The percentage of surfactant in the cooling composition may vary depending on the desired application of the cooling composition. As an example, the cooling composition may include about 1% to about 5% surfactant. In other embodiments, the cooling composition may include about 1% to about 4%, or about 1% to about 3%, or about 1% to about 2% surfactant. It should be noted that while the ingredients in the cooling composition may typically include about 1% to about 5% surfactant, some variability in the types of surfactant employed within the cooling composition is acceptable so long as the surfactant provides sufficient emulsifying activity.

It should be noted that in order to achieve solubilization or emulsification of a lipophilic ingredient, the lipophilic ingredient (e.g., an oil soluble skin health benefit agent) must be compatible with a surfactant that is part of the cooling composition in order to obtain a stable formulation. As examples, surfactants may be selected from groups of sorbitan fatty acids (sorbitan monopalmitate, sorbitan monolaurate and the like), polyoxyethylene sorbitan fatty acid esters (polyoxyethylene 20 sorbitan monolaurate, polyoxyethylene sorbitan 20 monostearate, polyoxyethylene 4 sorbitan monostearate and the like), polyoxyethylene acids (polyoxyethylene 8 stearate, polyoxyethylene 20 stearate and the like), and polyoxyethylene alcohols (polyoxyethylene 4 lauryl ether, polyoxyethylene 10 cetyl ether, polyoxyethylene 10 stearyl ether, polyoxyethylene 5.5 decyl ether, and the like).

The percentage of silicone in the cooling composition may vary depending on the desired application of the cooling composition. As an example, the cooling composition may include about 1% to about 5% silicone. In other embodiments, the cooling compositions may include about 1% to about 4%, or about 1% to about 3%, or about 1% to about 2% silicone. Therefore, while the ingredients in the cooling compositions may typically include about 1% to about 5% silicone, some variability in the types of silicone employed within the cooling compositions is acceptable so long as the cooling compositions provide sufficient immediate and long term cooling of the skin.

Suitable PEG dimethicones include PEG-3 Dimethicone, PEG-7 Dimethicone, PEG-8 Dimethicone, PEG-9 Dimethicone, PEG-10 Dimethicone, PEG-12 Dimethicone and PEG-14 Dimethicone (among others).

Polymeric Emulsifier Composition

In other embodiments, the cooling composition includes a polymeric emulsifier that is a cross linked acrylic acid polymer containing alkyl groups and/or acrylamide groups which aid in compatibility with organic oils. The cross-linking agent may be present in the cooling composition in such an amount as to provide enough adhesion between skin and the cooling composition to allow the desired cooling of skin to take place.

As an example, polymeric emulsifiers may be used in emulsifying and stabilizing emulsions containing up to 70% oil. Some polymeric emulsifiers may need to be neutralized in order to provide emulsifying activity.

Polymeric emulsifiers may be identified with an International Nomenclature Cosmetic Ingredient (INCI) identifier such as Acrylates/C10-30 Alkyl Acrylates Crosspolymer. Some specific example polymeric emulsifiers are Pemulen TR-1 and Pemulen TR-2 manufactured by Noveon Inc., 9911 Brecksville Road, Cleveland, Ohio 44141. Pemulen TR-1 and TR-2 are each a cross linked acrylic acid polymer containing alkyl groups which aid in compatibility with organic “oils” such that they are able to function as an emulsion stabilizer or polymeric emulsifier.

Other example polymeric emulsifiers include Novemer EC-1 manufactured by Noveon Inc., 9911 Brecksville Road, Cleveland, Ohio 44141. Novemer EC-1 is a cross linked acrylic acid polymer containing acrylamide groups which aid in compatibility with organic “oils” such that they are able to function as an emulsion stabilizer or polymeric emulsifier.

The percentage of polymeric emulsifier in the cooling composition may vary depending on the desired application of the cooling composition. As an example, the cooling composition may include about 1% to about 5% polymeric emulsifier. In other embodiments, the cooling compositions may include about 1% to about 4%, or about 1% to about 3%, or about 1% to about 2% polymeric emulsifier.

The cooling composition further includes water that may provide the cooling compositions with some long term cooling of the skin through the latent heat of evaporation. In some embodiments, the water may be purified or distilled.

The percentage of water in the cooling composition may vary depending on the desired application of the cooling composition. As an example, the cooling composition may include about 80% to about 95% water. In other embodiments, the cooling composition may include about 90% to about 95% water.

The cooling composition further includes a cooling agent that produces a perception of an immediate and/or long term cooling sensation on skin when the material is applied to skin. Suitable cooling agents include but are not limited to menthol, mentyl lactate, methyl salicylate, menthyl carbinal, mint, mint oils, cucumber, chamomile, aloe, comfrey, anise, sage, carboamides and ketals.

The percentage of cooling agent in the cooling composition may vary depending on the desired application of the cooling composition. As an example, the cooling composition may include about 1% to about 5% cooling agent. In other embodiments, the cooling composition may include about 1% to about 4%, or about 1% to about 3%, or about 1% to about 2% cooling agent. It should be noted that there may be some variability in the types of cooling agent employed within the cooling composition as long as the cooling agent provides sufficient short and long term skin cooling.

In some embodiments, the polymeric emulsifier may be utilized by dispersing the polymeric emulsifier in water such that the polymeric emulsifier swells. The swollen polymeric emulsifier may be neutralized with a minor amount (<1%) of a water soluble alkaline material (or some other neutralizer) such that the neutralized polymeric emulsifier forms a network that holds oil droplets in place. Some suitable alkaline neutralizers include sodium hydroxide, potassium hydroxide, ammonium hydroxide, dispropanolamine, sodium borate, sodium citrate, tetrahydoxypropyl ethylenediamine, triethanolamine, aminomethyl propanol and sodium glucamate (among others).

The network prevents each oil droplet from coalescing with another oil droplet such that the polymeric emulsifier provides emulsion stability. As an example, the polymeric emulsifier may form an adsorbed gel layer around each oil droplet with hydrophobic (alkyl groups) portions of the polymer anchored in the oil.

Depending on the type of polymeric emulsifier that is used in the cooling composition, proper neutralization of the polymer may be necessary to obtain viscosity control and stability of the cooling composition. Inadequate neutralization of the polymer may form a cooling composition that is unstable and/or have too low of a viscosity. In addition, over neutralization of the polymer may also result in an unstable cooling composition.

When the polymer is properly neutralized the cooling composition may be formed on a non-woven, fiber basesheet or a woven basesheet (e.g., a wipe). The hydrophobic portions (i.e., alkyl groups) of the formed network hold and separate oil droplets that include the cooling agent. Therefore, the network prevents at least some of the cooling agent from being absorbed by the natural or polymer fibers that form the wipe.

In some embodiments, the cooling composition may have shear thinning rheology due to the presence of the polymeric emulsifier (e.g., Pemulen TR-2). “Shear thinning” means that when shear is applied to the cooling composition, the viscosity of the cooling composition drops considerably.

As an example, the cooling composition is subjected to shear when the cooling composition is pumped through orifices in hoses and application heads as part of applying the cooling composition to a basesheet. In some embodiments, the shear may cause the cooling composition to thin to a very low viscosity (300 cps or less).

The cooling composition may become thin enough to thoroughly wet and uniformly impregnate a basesheet when the cooling composition is applied to the basesheet. After a short period of time, the viscosity of the cooling composition rebounds to the original viscosity such that the cooling composition is stabilized on the basesheet. The shear thinning property of the cooling composition may also facilitate transferring the cooling composition to skin because the cooling composition may thin to a lower viscosity as the cooling agent impregnated basesheet is rubbed on the skin.

In some embodiments, the cooling composition may also “break” upon application to the skin to facilitate transferring the cooling composition to the skin. This breaking (i.e., destabilization) of the cooling composition may be caused by natural salt content that is common on skin. The salt causes the acrylic hydrophilic portion of the polymer hydrogel to instantaneously “de-swell.” This de-swelling releases the cooling agent such that the cooling agent contacts the skin.

Therefore, while the cooling composition may include about 1% to about 5% polymeric emulsifier, some variability in the types of polymeric emulsifier employed within the cooling composition is acceptable so long as the cooling composition has a desired viscosity.

Silicone and Polymeric Emulsifier Composition

In some embodiments, the cooling composition includes a water dispersible silicone. As used herein, a water dispersible silicone includes water dispersible silicones and/or water soluble silicones. One example water dispersible silicone includes ethoxylated dimethicone (PEG (X) dimethicone) that conforms to the structure: 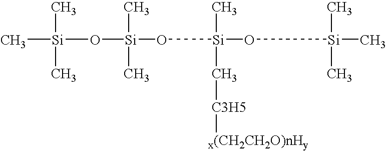
Where x=1 to about 500, y=1 to about 100 and n=1 to about 30.

The percentage of silicone in the cooling composition may vary depending on the desired application of the cooling composition. As an example, the cooling composition may include about 1% to about 5% silicone. In other embodiments, the cooling composition may include about 1% to about 4%, or about 1% to about 3%, or about 1% to about 2% silicone. It should be noted that while the cooling composition may include about 1% to about 5% silicone, some variability in the types of silicone employed within the cooling composition is acceptable so long as the cooling composition provides sufficient short and long term skin cooling. Some example PEG dimethicones include PEG-3 Dimethicone, PEG-7 Dimethicone, PEG 8 Dimethicone, PEG-9 Dimethicone, PEG-10 Dimethicone, PEG-12 Dimethicone and PEG-14 Dimethicone (among others).

The cooling composition further includes water and alcohol that provide the cooling composition with some of the short and long term cooling of the skin through the latent heat of evaporation from the alcohol (shorter term cooling) and water (longer term cooling). In some embodiments, the water may be purified or distilled. In addition, the alcohol may be isopropyl alcohol or ethyl alcohol.

The percentage of water and alcohol in the cooling composition may vary depending on the desired application of the cooling composition. As an example, the cooling composition may include about 1% to about 5% water. In other embodiments, the cooling composition may include about 1% to about 4%, or about 1% to about 3%, or about 1% to about 2% water. In addition, the cooling composition may include about 1% to about 5% alcohol. In other embodiments, the cooling composition may include about 1% to about 4%, or about 1% to about 3%, or about 1% to about 2% alcohol.

The cooling composition further includes a cooling agent that produces a perception of an immediate and/or long term cooling on skin when the cooling composition is applied to skin. The cooling that is provided by the cooling agent is in addition to the cooling provided by the alcohol and water through the latent heat of evaporation. Some example cooling agents include menthol, mentyl lactate, methyl salicylate, peppermint oil, oil of wintergreen, and menthyl carbinal, carboamides and ketals (among others). Any of the cooling agents described herein may be used in the silicone and polymeric emulsifier cooling composition.

The cooling composition further includes a polymeric emulsifier that is a cross linked acrylic acid polymer containing alkyl groups and/or acrylamide groups which aid in compatibility with organic oils such that they are able to function as an emulsion stabilizer or polymeric emulsifier. The percentage of polymeric emulsifier in the cooling composition may vary depending on the desired application of the cooling composition. As an example, the cooling composition may include about 1% to about 5% polymeric emulsifier. In other embodiments, the cooling compositions may include about 1% to about 4%, or about 1% to about 3%, or about 1% to about 2% polymeric emulsifier.

As discussed above, some of the polymeric emulsifiers that may be used in the cooling composition are identified with an International Nomenclature Cosmetic Ingredient (INCI) identifier such as Acrylates/C10-30 Alkyl Acrylates Crosspolymer (e.g., Pemulen TR-1, TR-2 and Novemer EC-1). As an example, polymeric emulsifiers may be used in emulsifying and stabilizing emulsions containing up to 70% oil. In addition, some polymeric emulsifiers may need to be neutralized in order to provide emulsifying activity.

In some embodiments, the polymeric emulsifier may be utilized by dispersing the polymeric emulsifier in water such that the polymeric emulsifier swells. The swollen polymeric emulsifier may be neutralized with a minor amount (<1%) of a water soluble alkaline material (or some other neutralizer) such that the neutralized polymeric emulsifier forms a network that holds oil droplets in place. Some suitable alkaline neutralizers include sodium hydroxide, potassium hydroxide, ammonium hydroxide, dispropanolamine, sodium borate, sodium citrate, tetrahydoxypropyl ethylenediamine, triethanolamine, aminomethyl propanol and sodium glucamate (among others).

In some embodiments, the cooling composition may have shear thinning rheology that facilitates applying the composition to a basesheet due to the presence of the polymeric emulsifier within the cooling composition. The shear thinning property of the cooling composition may also facilitate transferring the cooling composition from the basesheet to skin. The cooling composition may also break (i.e., destabilize) upon application to the skin to further facilitate transferring the cooling composition to skin.

Therefore, while the cooling composition may include about 1% to about 5% polymeric emulsifier, some variability in the types of polymeric emulsifier that are within the cooling composition is acceptable so long as the cooling composition maintains a desired viscosity.

In some embodiments, the cooling composition may further include a surfactant that acts as an emulsifying agent and/or solubilizing agent. Any of the surfactants previously described herein may be included in the cooling composition. The surfactants may have an HLB of about 6 to about 18. In addition, a combination of surfactants may be used to achieve a desired HLB.

The percentage of surfactant in the cooling composition may vary depending on the desired application of the cooling composition. As an example, the cooling composition may include about 1% to about 5% surfactant. In other embodiments, the cooling composition may include about 1% to about 4%, or about 1% to about 3%, or about 1% to about 2% surfactant.

Administration

The cooling composition may be administered directly to the skin for prophylactic, therapeutic and/or hygienic use. The cooling composition may be administered in a single dose, in multiple doses and/or in a continuous or intermittent manner depending on a variety of factors (e.g., the recipient's physiological condition).

The cooling composition may be formulated into a variety of articles (e.g., patches, bandages, sponges, wipes and dressings). In addition, the viscosity of the cooling composition may be controlled in part by adding other items to the cooling composition.

The cooling composition may be administered in the form of a stick, powder/talc or other solid, solution, liquid, spray, bioadhesive gel, aerosol, foam, cream, gel, lotion, paste, jellies or sprays. As an example, creams may be formulated with an aqueous or oily base with additional suitable thickening and/or gelling agents. In addition, lotions may be formulated with additional stabilizing agents, dispersing agents, suspending agents, thickening agents, or coloring agents.

In some embodiments, the cooling composition may degrade slowly and remain attached to the skin for a period of time. As an example, the cooling composition may include a bioadhesive that has a cross-linking agent which is present to facilitate adhering the cooling composition to the skin. The bioadhesive may be included at various concentrations within the cooling composition in order to provide more or less adhesion to the skin.

When the cooling composition is a liquid, the cooling composition may be administered from absorbent materials (e.g., a bandage or sponge). The cooling composition may also be administered as a spray/aerosol that is applied to the skin using a pump-type or aerosol sprayer. In some embodiments, the cooling composition may be provided in the form of a solution that is initially in the form of a concentrated liquid, dissolvable powder or tablet where water, saline or other liquid is added to form the cooling composition prior to use.

The cooling composition may also be administered using an applicator (e.g., a squeeze-type or plunger-type applicator). In some embodiments, the cooling composition may be administered as a cream that has low surface tension to provide a uniform wetting action as the cooling composition is applied to the skin. It should be noted that other delivery vehicles for applying the cooling composition to the skin are contemplated and within the scope of the present invention.

Additives

The cooling composition may include other active or inactive additives depending on the desired application of the cooling composition. It should be noted that the absolute weight of any additive to the cooling composition may vary.

In some embodiments, the cooling composition may include therapeutic additives. Some example therapeutic additives include anti-microbial agents, pain relievers, anti-inflammatory agents, skin-protectants, antiseptics, sunscreens, insect repellents, exfoliants, deodorants, antiperspirant vitamins (e.g., vitamin B, C or E) and aloe vera (among others).

The cooling composition may also include an additive that regulates the release of one or more of the items which form the cooling composition at a desired rate. As an example, the additive may provide for long term delivery of one or more items in the cooling composition thus increasing the useful life of a product that includes the cooling composition. The appropriate amount of such an additive will depend on the desired rate and duration of the release. Examples of such additives include water insoluble polymers such as ethylcellulose, acrylic resins, co-polymer of methacrylic acid and acrylic acid ethyl ester, polylactic acid, PLGA, polyurethane, polyethylene vinyl acetate copolymer and polystyrene-butadiene copolymer (or mixtures thereof).

There are other additives that may be included in the cooling composition to facilitate delivering the cooling composition to skin. Some examples of such additives include lubricants, plasticizing agents, preservatives, thickeners, emulsion stabilizers, stick formers, suppository formers, film formers, cream formers, coatings, binders, carrier, coloring agents, moisturizers, chelating agents, fragrance and/or odor controlling agents, humectants, viscosity controlling agents and pH-adjusting agents (among others).

It should be noted that any number and type of additives may be included in the cooling composition. Some of the other example additives include potassium lactate, vitamin E, vitamin C, fragrance, botanicals, citric acid, sodium hydroxide and/or potassium chloride (among others).

Methods of Forming Silicone Cooling Composition

Some embodiments of the invention are directed to methods of forming a silicone cooling composition. The methods produce skin cooling compositions that provide that provide immediate and long term skin cooling.

The method includes mixing water and alcohol to form a first mixture and mixing a solubilizing surfactant with vitamin E and a water dispersible silicone to form a second mixture. The method further includes adding a derivative of lactic acid to the first mixture to form a third mixture and mixing the third mixture with the second mixture to form the cooling composition. The method may further include adjusting the pH of the cooling composition to about 5.5 with citric acid.

In some embodiments, mixing a solubilizing surfactant with vitamin E and silicone to form a second mixture may include heating the second mixture to disperse the silicone within the second mixture. In addition, mixing the third mixture with the second mixture to form the cooling composition may include slowly titrating the second mixture into the third mixture.

In some embodiments, mixing a solubilizing surfactant with vitamin E and silicone to form a second mixture may include mixing polysorbate 20 with tocopherol acetate and silicone glycol. It should be noted that the method may further includes adding other materials (e.g., natural oils, other oil soluble vitamins and esters) to the second mixture. In addition, adding a derivative of lactic acid to the first mixture to form a third mixture may include adding potassium lactate to the first mixture.

Methods of Forming Polymeric Emulsifier Cooling Composition

Some embodiments of the invention are directed to methods of forming a polymeric emulsifier cooling composition. The methods produce skin cooling compositions that provide that provide immediate and long term skin cooling.

The method includes mixing vitamin C with a derivative of lactic acid and a solubilizing surfactant to form a first mixture and mixing a cooling agent with vitamin E and a polymeric emulsifier to form a second mixture. The method further includes mixing the first mixture with the second mixture to form a third mixture and mixing water with a neutralizing base and a preservative to form a fourth mixture. The method further includes mixing the fourth mixture with the third mixture to form the cooling composition.

The method may further include adjusting the pH of the cooling composition to about 5.5 (e.g., by adding TEA among other items), and/or mixing together fragrance and another solubilizing surfactant (e.g., polysorbate 20) to form a fifth mixture that is added to the cooling composition.

In some embodiments, mixing water with a neutralizing base and a preservative to form a fourth mixture may include separately mixing the water, the neutralizing base and the preservative. In addition, mixing the fourth mixture with the third mixture to form the cooling composition may include slowly adding the fourth mixture to the third mixture to increase viscosity of the cooling composition.

In some embodiments, mixing vitamin C with a derivative of lactic acid and a solubilizing surfactant to form a first mixture may include (i) heating the first mixture to 85° C.; and/or (ii) mixing vitamin C with potassium lactate and polysorbate 20. In addition, mixing a cooling agent with vitamin E and a polymeric emulsifier to form a second mixture may include (i) heating the second mixture to 72° C.; (ii) dispersing the polymeric emulsifier throughout the second mixture; and/or (iii) mixing Finsolv TN and Frescolat ML with vitamin E and Pemulin TR-2.

In some embodiments, mixing the first mixture with the second mixture to form a third mixture may include mixing the first mixture with the second mixture using shear agitation. As an example, mixing the first mixture with the second mixture using shear agitation may include mixing the first mixture with the second mixture using a high shear agitation for about 2 minutes and then mixing the first mixture with the second mixture using a lower shear agitation for about 15 minutes.

The following Examples further illustrate certain aspects of the invention and are not intended to limit the invention in any manner.

### EXAMPLE #1

### Silicone Cooling Composition

This Example describes experiments showing the formation of one example silicone cooling composition that provides short and long term skin cooling.

Materials and Methods

Volume=0.250 L

| Ingredients | % wt | Grams | Trade Name |
|---|---|---|---|
| Phase A | | | |
| Water | Q.S. | 188.5000 | WRE 4-10-03 |
| SD Alcohol 40B | 20.00 | 50.0000 | SDA 40B 190 Proof |
| Phase B | | | |
| Potassium Lactate | 0.25 | 0.6250 | Potassium-L-Lactate |
| | | | HiPure 60 |
| Phase C | | | |
| Polysorbate 20 | 2.00 | 5.0000 | Tween 20 |
| Tocopherol Acetate | 0.10 | 0.2500 | di-alpha-Tocopheol |
| (Vit E) | | | Acetate |
| Silicone Glycol | 2.00 | 5.0000 | Ultrasil Copolyol 1 |
| Phase D | | | |
| Citric Acid | pH 5.5 | 0.0500 | 50% Citric Acid Solution |
| Total Before Water | 23.75 | 29.0625 | |
| Total After Water | 100.00 | −125.0500 | |

1) Mix all ingredients of Phase A.

2) Mix all ingredients of Phase C. Heat to melt and disperse silicone glycol.

3) Add Phase B to Phase A.

4) Slowly titrate Phase C into Phase A/B.

5) Adjust pH of suspension (Phase A/B/C) to 5.5 with Citric Acid (Phase D).

The polysorbate 20/vitamin E/silicone glycol mix stiffened up upon sitting. It was added to the water phase in a semi solid state. The pH was 6.750 before adding Phase B to Phase A/C. The pH was 5.715 after adding Phase B to Phase A/C. The pH was adjusted to 5.494 with 0.05 grams citric acid.

Results

The cooling composition that was created remained crystal clear and never became clouded during addition of ingredients. The cooling composition provided a cooling sensation without leaving a sticky feeling.

### EXAMPLE #2

### Polymeric Emulsifier Cooling Composition

This Example describes experiments showing the formation of one example polymeric emulsifier cooling composition that provides short and long term skin cooling.

Materials and Methods

Volume=0.125 L

| Ingredients | % wt | Grams | Trade Name |
|---|---|---|---|
| Phase A | | | |
| Water | Q.S. | 87.0875 | |
| Ascorbic Acid Ester | 0.50 | 0.6250 | Not added/not available |
| (Vit C) | | | |
| Potassium Lactate | 0.75 | 0.9375 | Potassium-L-Lactate |
| Polysorbate 20 | 2.00 | 2.5000 | Tween 20 |
| Tocopherol Acetate | 0.50 | 0.6250 | di-alpha-Tocopherol Acetate |
| (Vit E) | | | |
| Sodium Hydroxide | pH 5.5 | | Sodium Hydroxide, 1N |
| | | | Volumetric Solutions |
| Phase B | | | |
| Finsolv TN | 3.00 | 3.7500 | Finsolv TN |
| Frescolate | 1.00 | 1.2500 | Frescolat ML |
| Vitamin E | 0.10 | 0.1250 | |
| Pemulin TR-2 | 0.10 | 0.1250 | Pemulin TR-2 |
| Phase C | | | |
| Water | 20.00 | 25.0000 | |
| TEA | 0.08 | 0.1000 | Trimethanolamine 98% |
| Preservative | 1.50 | 1.8750 | Paragon G2 |
| Phase D | | | |
| Fragrance | 0.20 | 0.2500 | Not added |
| Polysorbate 20 | 0.60 | 0.7500 | Not added |
| Total Before Water | 30.33 | 37.9125 | |
| Total After Water | 100.00 | 125.000 | |

1) Mix all ingredients of Phase A. Heat to 85° C.

2) Mix together ingredients of Phase B. Heat to 72° C., dispersing the Pemulin throughout the mixture as the Pemulin will not solubilize.

3) Add Phase B to Phase A using high shear agitation. Mix for 2 minutes. Mix for another 15 minutes using less shear agitation so that the Pemulin swells and formulation cools.

4) Separately mix the ingredients of Phase C. Slowly add Phase C to Phase A/B to increase viscosity of the composition.

6) Adjust pH to 5.5 or greater using TEA. If the pH is higher than 5.5 do not adjust pH.

7) Mix together Fragrance and Polysorbate 20 and add to the composition.

Results

The example method produced a milky composition that readily mixed together. The composition appeared well suited for addition as a cooling agent to water base wet wipes products.

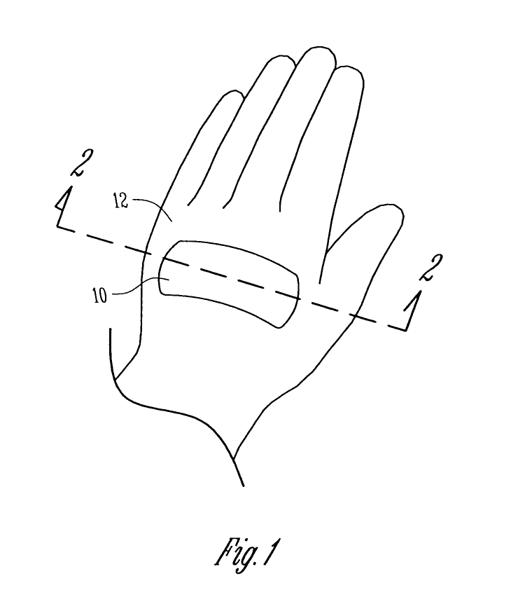

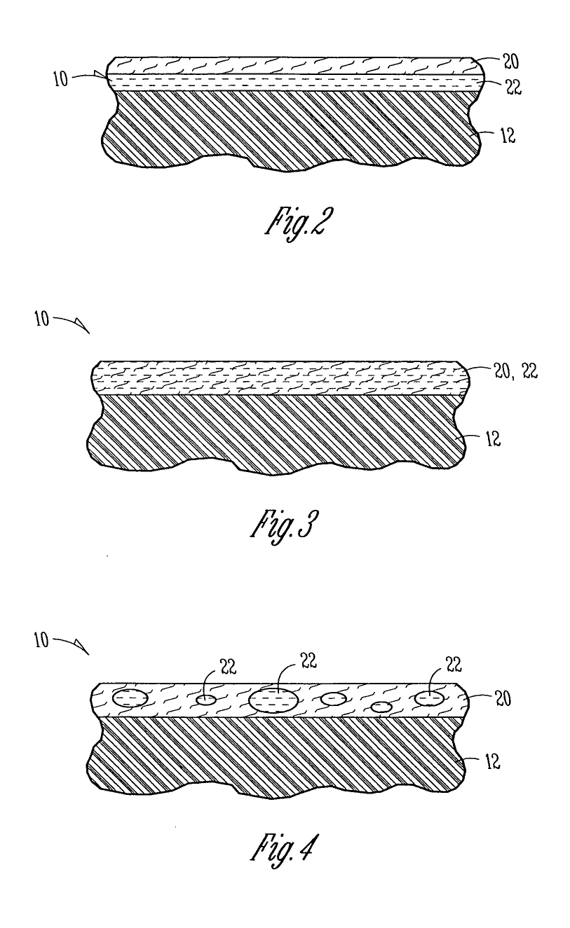

FIG. 1 illustrates an applicator 10 that is applied to skin 12 to cool skin 12. As shown in FIGS. 2-4, the applicator 10 includes a substrate 20 and a skin cooling composition 22 that cools skin 12. Any of the skin cooling compositions described herein may be used in the applicator 10.

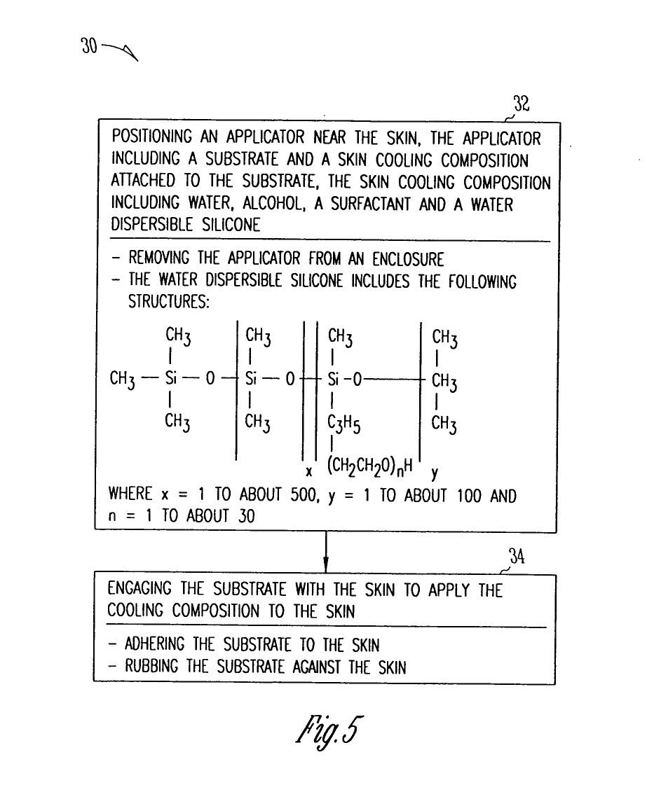

The skin cooling composition 22 is attached to (FIG. 2), or made part of (FIGS. 3 and 4), the substrate 20 through any method known now or discovered in the future. As used herein, “substrate” refers to any type of substrate including laminates of one or more types of material. The substrate 20 may also be formed of multiple layers of materials.

In the example embodiment illustrated in FIG. 2, substrate 20 and skin cooling composition 22 are formed as separate layers that are joined together. Although substrate 20 and skin cooling composition 22 are shown as layers of the same size, it should be noted that the layers may be different sizes and/or shapes.

Some example materials that may be used for substrate 20 include cotton, rayon, wood pulp and polymeric substances such as nonwoven fabrics, foam sponges and thermoplastics. It should be noted that substrate 20 may be designed with any type of surface (e.g., textured) that enhances the ability of the applicator 10 to apply the skin cooling composition 22 to skin 12. In addition, the substrate 20 may dissolve, disperse and/or evaporate in the presence of water, air, salt, heat, acidity or other items.

As used herein, “nonwoven fabric” refers to a web or fabric (made partly or wholly of non-cellulose material) that includes individual fibers which are interlaid (randomly or in a pattern) in a knitted fabric. The nonwoven webs may be formed by many processes, including meltblowing, spunbonding and bonded carded. The nonwoven fabrics that are used in the substrate 20 may be produced from polymers such as polyethylene or polypropylene.

The substrate 20 may also be formed of a nonwoven fabric that is made of an array of interbonded thermoplastic fibers. The fibers may be formed from a wide variety of thermoplastic materials. Some example thermoplastic materials include polyolefins (e.g., polyethylene, polypropylene), polystyrene and polyamides (e.g., nylon). In addition, thermoplastic polymers that are elastomeric may also be used as fibers. Some example elastomeric materials include polyurethanes and block copolymers. It should be noted that blends of any of the foregoing materials may be used to form the fibers.

The fibers may also include additives (e.g., wax, pigments, stabilizers and fillers) that are inserted as the fibers are fabricated to achieve one or more desired properties within the fibers. Some example additives include compatible surfactants that are added to the polymers to make the surface of the fibers more wettable.

In some embodiments, the substrate 20 may include a cover material (not shown) that is placed on the substrate 20 to prevent fiber sloughing. In addition, some cover materials may improve the wettablilty of the substrate 20. Some example cover materials include spunbound, spunlace, bonded carded web and apertured film materials. In one example embodiment, the cover material may be an apertured film that is formed of a polyolefin which may or may not be combined with a nonwoven fabric.

When a nonwoven fabric is used as the material for the substrate 20, the basis weight of the nonwoven fabric may vary depending on the properties that are desired within the applicator 10. As an example the basis weight for the nonwoven fabric may be as low as 10 grams per square meter and as high as 300 grams per square meter.

FIGS. 3 and 4 show that the skin cooling composition 22 may also be incorporated into substrate 20. FIG. 3 shows that the skin cooling composition 22 may be at least partially, or wholly, soluble in the substrate 20, while FIG. 4 shows that the skin cooling composition may be suspended in the substrate 20.

In some embodiments, the substrate 20 may tacky enough such that the substrate 20 is able to adhere the applicator 10 to skin 12. The skin cooling composition 22 may be made part of the substrate 20 through any method known now or discovered in the future.

A range of materials can be used to hold the skin cooling composition 22 and/or form the substrate 20 such that the skin cooling composition 22 is released over time. These materials may include one or more thermoplastics, organic polymers (e.g., natural polysaccharides), starches, gums, alginate and xanthan (among other materials). The quantity of skin cooling composition 22 in the applicator 10 will be based on the properties that are desired within particular commercial products.

In other embodiments, skin cooling composition 22 may be stored in a reservoir layer when the substrate 20 has a laminate structure. The laminate structure may include a rate limiting layer that releases the skin cooling composition 22 over time.

The skin cooling composition 22 may also be placed in a porous material (e.g., foam) that releases skin cooling composition 22 over time. The release rate of the skin cooling composition 22 will be determined in part by the nature of the porous material and the form of the skin cooling composition 22.

In alternative embodiments, the skin cooling composition 22 may be incorporated into a substance that dissolves away over time as the applicator 10 is exposed to skin. Therefore, as the substance dissolves, the skin cooling composition 22 may be released.

In some embodiments, the applicator 10 may further include a bonding material (not shown) that attaches the applicator 10 to skin 12. The bonding material 24 may be attached to the substrate 20 and/or skin cooling composition 22, or made part of the substrate 20 or skin cooling composition 22, through any method known now or discovered in the future.

As an example, the bonding material may be in the form of an adhesive layer that is attached to the substrate 20 and/or skin cooling composition 22. The applicator 10 may be attached to the skin 12 by adhering the adhesive layer to skin 12. In some embodiments, the adhesive layer may attach the applicator 10 to skin 12 until the applicator 10 is peeled off so that no adhesive or residue is left behind on skin 12.

It should be noted that the bonding material may be separate from the substrate 20 and skin cooling composition 22, or form part of the substrate 20 or skin cooling composition 22. In embodiments where the bonding material forms part of the substrate 20 and/or skin cooling composition 22, the bonding material may be (i) suspended in the substrate 20 and/or skin cooling composition 22; or (ii) soluble in the substrate 20 and/or skin cooling composition 22.

Some example materials that may be used for the bonding material include pressure-sensitive adhesives (PSAs), hotmelt adhesives, reactive adhesives (e.g., isocyanates, cyanoacrylates, acrylics, and ethylvinylacetates), latex adhesives and epoxies. The type of bonding material will be selected based on processing parameters, solubility and whether the bonding material forms part of the substrate 20 and/or skin cooling composition 22 (among other factors).

The applicator 10 may further include other ingredients (e.g., moisturizers and other products that benefit the skin 12). The applicator 10 may also include a fragrance.

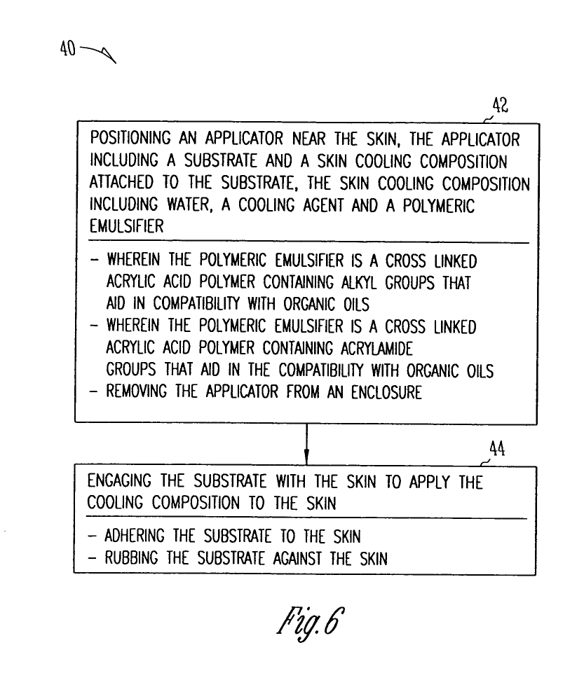

FIG. 5 shows an example method 30 of cooling skin. The method 30 includes positioning an applicator near the skin 32 where the applicator includes a substrate and a skin cooling composition that is attached to the substrate. The skin cooling composition includes water, alcohol, a surfactant and a water dispersible silicone. In some embodiments, positioning an applicator near the skin 32 may include removing the applicator from an enclosure (e.g., an air-tight package).

The method 30 further includes engaging the substrate with the skin to apply the skin cooling composition to the skin 34. In some embodiments, engaging the substrate with the skin 34 includes adhering the substrate to the skin. It should be noted that engaging the substrate with the skin 34 may also include rubbing the substrate against the skin.

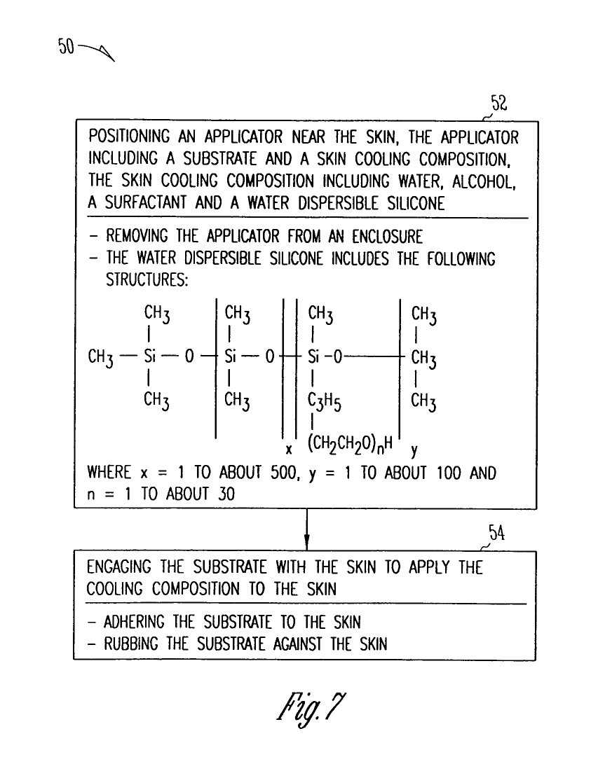

FIG. 6 shows another example method 40 of cooling skin. The method 40 includes positioning an applicator near the skin 42 where the applicator includes a substrate and a skin cooling composition that is attached to the substrate. The skin cooling composition includes water, a cooling agent and a polymeric emulsifier. In some embodiments, positioning an applicator near the skin 42 may include removing the applicator from an enclosure (e.g., an air-tight enclosure).

The method 40 further includes engaging the substrate with the skin to apply the skin cooling composition to the skin 44. In some embodiments, engaging the substrate with the skin 44 includes adhering the substrate to the skin and/or rubbing the substrate against the skin.

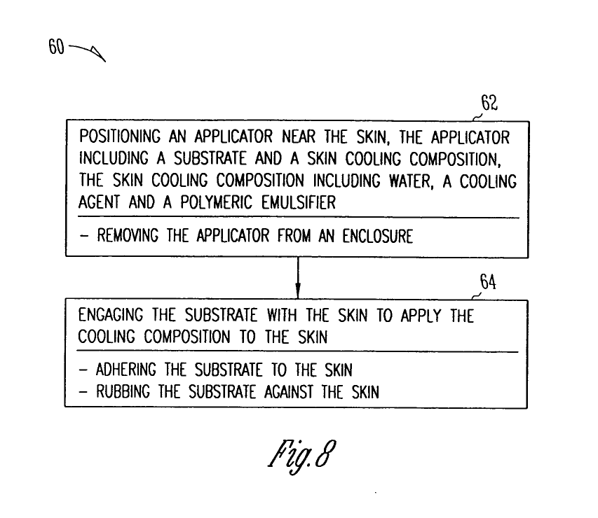

FIG. 7 shows still another example method 50 of cooling skin. The method 50 includes positioning an applicator near the skin 52 where the applicator includes a substrate having a skin cooling composition. The skin cooling composition includes water, alcohol, a surfactant and a water dispersible silicone.

The method 50 further includes engaging the substrate with the skin to apply the skin cooling composition to the skin 54. In some embodiments, engaging the substrate with the skin 54 may include adhering the substrate to the skin. It should be noted that engaging the substrate with the skin 54 may also include rubbing the substrate against the skin.

FIG. 8 shows yet another example method 60 of cooling skin. The method 60 includes positioning an applicator near the skin 62 where the applicator includes a substrate having a skin cooling composition. The skin cooling composition includes water, a cooling agent and a polymeric emulsifier.

The method 60 further includes engaging the substrate with the skin to apply the cooling composition to the skin 64. In some embodiments, engaging the substrate with the skin 64 may include adhering the substrate to the skin and/or rubbing the substrate against the skin.

As part of fabricating the applicator 10, multiple applicators may be cut from a sheet that is feed out from a continuous roll (e.g., a perforated roll). The multiple applicators may then be stacked for packaging or delivered as the continuous roll. In some forms, multiple applicators may be inter-folded, o-folded and/or compressed into various geometric shapes. In addition, when the applicators are stacked, the applicators may be separated by a barrier that is situated between the applicators. In some embodiments, the applicator 10 may be embossed with logos, use instructions or any other design or information.

The type of substrate 20 and skin cooling composition 22 that is used in the applicator 10 will be selected based on (i) processing parameters; (ii) dispensing characteristics; (iii) packaging issues; and (iv) solubility. In addition, the properties of the substrate 20 and the skin cooling composition 22 may be controlled to define the shear strength, viscosity and/or heat stability (among other properties) of the applicator 10.

It should be noted that the applicator 10 may be in any form or shape that cools a particular area. As an example, the applicator 10 may be in the form of a continuous strip. The applicator 10 may also be decorative in color and/or shape depending on consumer appeal.

Other embodiments are contemplated where the applicator 10 has a clear and/or unobtrusive product form such that the applicator 10 discreet. In addition, the applicator 10 may include an indicator (e.g., a section that changes color) which provides information as to when the applicator 10 is out of skin cooling composition 22 and requires replacement.

The embodiments and Examples described herein are representative and are not intended as limitations on the scope of the invention. Although the invention has been disclosed with reference to some example embodiments, modifications and variations are considered to be within the scope of the invention as defined by the appended claims.

The invention described herein may be practiced in the absence of any element or limitation which is not specifically disclosed herein as essential. In addition, the methods and processes described herein may be performed in differing orders such that they are not necessarily restricted to the order described herein.

What is claimed is:

1. An applicator comprising:
a substrate; and
a skin cooling composition attached to the substrate, the skin cooling composition including water, alcohol, a surfactant and a water dispersible silicone.

2. The applicator of claim 1, wherein the water dispersible silicone includes the following structures: 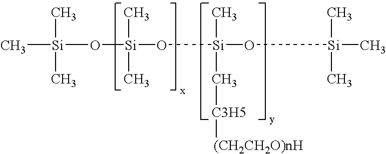
Where x=I to about 500, y=1 to about 100 and n=I to about 30.

3. The applicator of claim 1, wherein an amount of the alcohol in the skin cooling composition is between 15% and 45% by weight based on a total amount of the skin cooling composition.

4. The applicator of claim 1, wherein an amount of the surfactant in the skin cooling composition is between 1% and 5% by weight based on a total amount of skin cooling composition.

5. The applicator of claim 1, wherein an amount of the silicone in the skin cooling composition is between 1% and 5% by weight based on a total amount of the skin cooling composition.

6. The applicator of claim 1, wherein the substrate is a polymer matrix.

7. The applicator of claim 1, wherein the substrate dissolves upon contact with skin.

8. The applicator of claim 1, wherein the skin cooling composition is suspended in the substrate.

9. The applicator of claim 1, wherein the skin cooling composition is at least partially soluble in the substrate.

10. The applicator of claim 1, wherein the substrate is a nonwoven fabric.

11. An applicator comprising:
a substrate; and
a skin cooling composition attached to the substrate, the skin cooling composition including water, a cooling agent and a polymeric emulsifier.

12. The applicator of claim 11, wherein an amount of the cooling agent in the cooling composition is between 1% and 5% by weight based on a total amount of the cooling composition.

13. The applicator of claim 11, wherein an amount of the polymeric emulsifier in the cooling composition is between 1% and 5% by weight based on a total amount of cooling composition.

14. The applicator of claim 11, wherein the polymeric emulsifier is a cross linked acrylic acid polymer containing alkyl groups that aid in compatibility with organic oils.

15. The applicator of claim 11, wherein the polymeric emulsifier is a cross linked acrylic acid polymer containing acrylamide groups that aid in compatibility with organic oils.

16. The applicator of claim 11, wherein the substrate is a polymer matrix.

17. The applicator of claim 11, wherein the substrate dissolves upon contact with skin.

18. The applicator of claim 11, wherein the skin cooling composition is suspended in the substrate.

19. The applicator of claim 11, wherein the skin cooling composition is at least partially soluble in the substrate.

20. The applicator of claim 11, wherein the substrate is a nonwoven fabric.

21. The applicator of claim 11, wherein the skin cooling composition further comprises a water soluble alkaline material that neutralizes the polymeric emulsifier.

22. An applicator comprising:
a substrate that includes a skin cooling composition, the skin cooling composition including water, alcohol, a surfactant and a water dispersible silicone.

23. The applicator of claim 22, wherein the substrate adheres to skin.

24. The applicator of claim 22, wherein the water dispersible silicone includes the following structures: 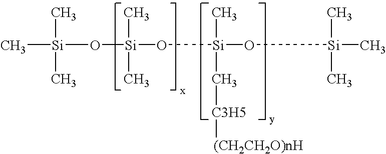
Where x=1 to about 500, y=1 to about 100 and n=1 to about 30.

25. The applicator of claim 22, wherein the substrate dissolves upon contact with skin.

26. The applicator of claim 22, wherein the skin cooling composition is suspended in the substrate.

27. The applicator of claim 22, wherein the skin cooling composition is at least partially soluble in the substrate.

28. An applicator comprising:
a substrate that includes a skin cooling composition, the skin cooling composition including water, a cooling agent and a polymeric emulsifier.

29. The applicator of claim 28, wherein the substrate adheres to skin.

30. The applicator of claim 28, wherein the polymeric emulsifier is a cross linked acrylic acid polymer containing alkyl groups that aid in compatibility with organic oils.

31. The applicator of claim 28, wherein the polymeric emulsifier is a cross linked acrylic acid polymer containing acrylamide groups that aid in compatibility with organic oils.

32. The applicator of claim 28, wherein the substrate dissolves upon contact with skin.

33. The applicator of claim 28, wherein the skin cooling composition is suspended in the substrate.

34. The applicator of claim 28, wherein the skin cooling composition is at least partially soluble in the substrate.

35. A method of cooling skin, the method comprising:
positioning an applicator near the skin, the applicator including a substrate and a skin cooling composition attached to the substrate, the skin cooling composition including water, alcohol, a surfactant and a water dispersible silicone; and
engaging the substrate with the skin to apply the cooling composition to the skin.

36. The method of claim 35, wherein the water dispersible silicone includes the following structures: 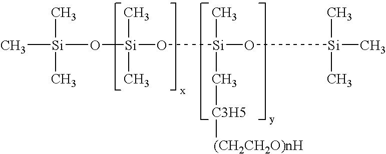
Where x=I to about 500, y=1 to about 100 and n=1 to about 30.

37. The method of claim 35, wherein engaging the substrate with the skin includes adhering the substrate to the skin.

38. The method of claim 35, wherein engaging the substrate with the skin includes rubbing the substrate against the skin.

39. The method of claim 35, wherein positioning an applicator near the skin includes removing the applicator from an enclosure.

40. A method of cooling skin, the method comprising:
positioning an applicator near the skin, the applicator including a substrate and a skin cooling composition attached to the substrate, the skin cooling composition including water, a cooling agent and a polymeric emulsifier; and
engaging the substrate with the skin to apply the cooling composition to the skin.

41. The method of claim 40, wherein the polymeric emulsifier is a cross linked acrylic acid polymer containing alkyl groups that aid in compatibility with organic oils.

42. The method of claim 40, wherein the polymeric emulsifier is a cross linked acrylic acid polymer containing acrylamide groups that aid in compatibility with organic oils.

43. The method of claim 40, wherein engaging the substrate with the skin includes adhering the substrate to the skin.

44. The method of claim 40, wherein engaging the substrate with the skin includes rubbing the substrate against the skin.

45. The method of claim 40, wherein positioning an applicator near the skin includes removing the applicator from an enclosure.

46. A method of cooling skin, the method comprising:
positioning an applicator near the skin, the applicator including a substrate having a skin cooling composition, the skin cooling composition including water, alcohol, a surfactant and a water dispersible silicone; and
engaging the substrate with the skin to apply the cooling composition to the skin.

47. The method of claim 46, wherein the water dispersible silicone includes the following structures: 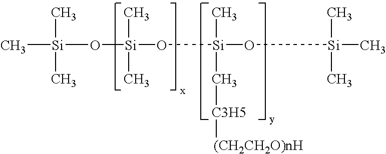
Where x=1 to about 500, y=1 to about 100 and n=1 to about 30.

48. The method of claim 46, wherein engaging the substrate with the skin includes adhering the substrate to the skin.

49. The method of claim 46, wherein engaging the substrate with the skin includes rubbing the substrate against the skin.

50. The method of claim 46, wherein positioning an applicator near the skin includes removing the applicator from an enclosure.

51. A method of cooling skin, the method comprising:
positioning an applicator near the skin, the applicator including a substrate having a skin cooling composition, the skin cooling composition including water, a cooling agent and a polymeric emulsifier; and
engaging the substrate with the skin to apply the cooling composition to the skin.

52. The method of claim 51, wherein engaging the substrate with the skin includes adhering the substrate to the skin.

53. The method of claim 51, wherein engaging the substrate with the skin includes rubbing the substrate against the skin.

54. The method of claim 51, wherein positioning an applicator near the skin includes removing the applicator from an enclosure.
